(19) United States
(12) Patent Application Publication
(10) Pub. No.: US 2005/0113651 A1
(43) Pub. Date: May 26, 2005

(54) Apparatus and method for surgical planning and treatment monitoring

(75) Inventors: Chris H. Wood, North Bend, WA (US); James J. Boisseranc, Snohomish, WA (US)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Confirma, Inc., Kirkland, WA (US)

(21) Appl. No.: 10/993,701

(22) Filed: Nov. 19, 2004

Related U.S. Application Data
(60) Provisional application No. 60/525,576, filed on Nov. 26, 2003.

(51) Int. Cl. G06F 17/60; A61B 5/00

(52) U.S. Cl. 600/300; 705/3

(57) ABSTRACT

A system for surgical planning and therapeutic monitoring utilizes imaging data and computer-aided detection (CAD) technology to identify cancerous tumors. A pre-treatment report identifies all volumes of interest (VOIs) and provides data regarding the size and location of each VOI as well as volumetric data for use in surgical planning. The system can be used to monitor the progress of adjuvant chemotherapy or other non-surgical treatment and measures changes in tumor size and location. Post-treatment reports provide data regarding changes in tumor size and location as well as trend data to provide guidance to the physician.

### BACKGROUND OF THE INVENTION

1. Field of the Invention

The present invention is directed generally to techniques for surgical planning and, more particularly, to an apparatus and method for surgical planning and treatment monitoring using medical imaging techniques.

2. Description of the Related Art

Breast cancer affects millions of individuals. In addition to breast self-examination, current medical advice includes periodic mammograms, which utilize conventional X-ray technology. If lesions or tumors are discovered, the X-ray or mammogram is used to identify and locate the region.

Conventional procedures for treatment include radiation and/or chemotherapy as well as surgical removal of the lesion. The surgical procedure may range from a lumpectomy to a mastectomy. Drug and radiation treatments are sometimes used pre-operatively to reduce or shrink the tumor size.

In a typical lumpectomy, the surgeon uses X-ray to identify the region containing the tumor and removes a large area surrounding the tumor. Unfortunately, this procedure often results in positive margins. That is, margins or regions bordering the removed tissue test positive for cancer and require additional surgery. Using current technology, up to 70% of lumpectomies result in positive margins that require additional surgery.

Therefore, it can be appreciated that there is a significant need for techniques to allow surgical planning, and pre-operative and post-operative treatment monitoring. The present invention provides this and other advantages as will be apparent from the following detailed description and accompanying figures.

### BRIEF DESCRIPTION OF THE SEVERAL VIEWS OF THE DRAWING(S)

FIG. 1 is a functional block diagram of a system constructed in accordance with the present teachings.

FIG. 2 is a flow chart illustrating operation of the system of FIG. 1.

FIG. 3 is a graphical image of a volume of interest identified as a possible tumor.

FIG. 4 is a magnetic resonance imaging MRI coronal image of breasts and anatomical location indicators.

FIG. 5 illustrates multiple MRI views of breasts and identification of the chest wall within the images.

FIG. 6 illustrates a MRI transverse image in which the skin surface is identified in three dimensions.

FIG. 7 illustrates computer modeling of regions or volumes of interest for surgical planning purposes.

FIG. 8 illustrates a pre-treatment report, including MRI image, of breasts, with anatomical features identified and anatomical data and measurements displayed.

FIG. 9 illustrates a post-treatment report, including identified anatomical features and volumes of interest and data related to pre- and post-treatment measurements.

FIG. 10 is an illustration of post-treatment reports indicating trends in treatment.

FIG. 11 illustrates breast imaging techniques with wire frame modeling of breasts and regions or volumes of interest.

FIG. 12 is an enlarged view of a portion of FIG. 11 illustrating a region of interest and a wire frame of a surrounding ellipsoid.

### DETAILED DESCRIPTION OF THE INVENTION

As will be discussed in further detail, the system described herein is directed to techniques for cataloging and measuring lesions or volumes of interest (VOI) for purposes of surgical planning and treatment monitoring. Although the techniques discussed herein use examples directed to evaluation of breast tumors, the techniques are more widely applicable to the evaluation of tissue for surgical planning purposes in general.

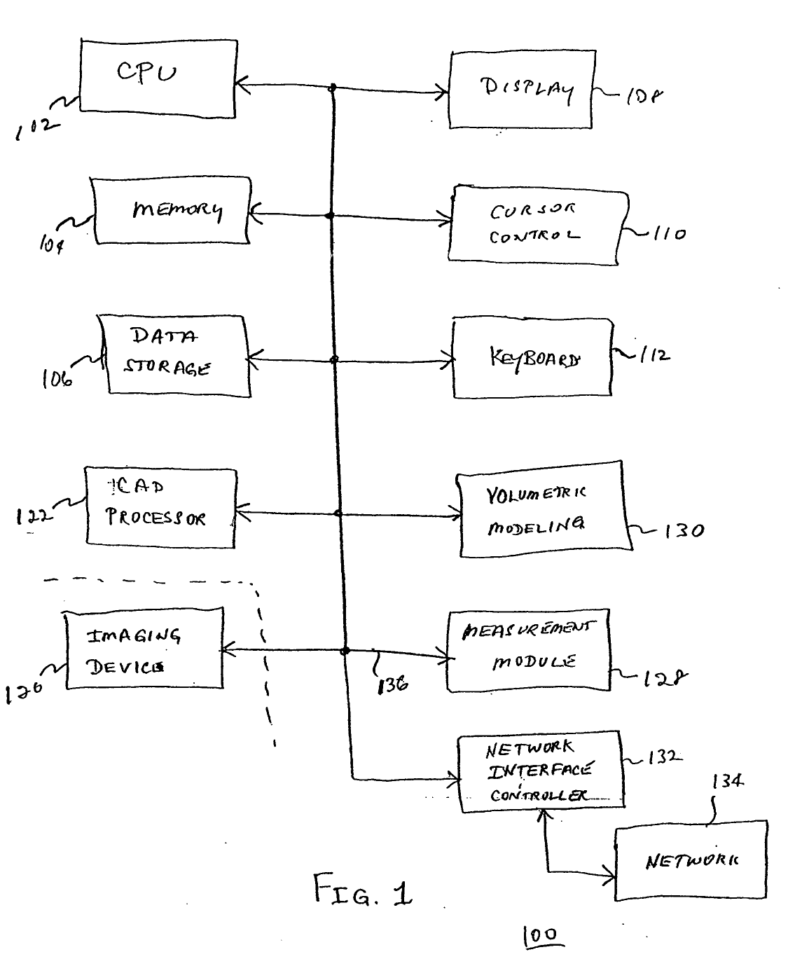

FIG. 1 is a functional block diagram of a system 100 constructed in accordance with the principles described herein. Many of the components of the system 100 are implemented as conventional computer components and need only be described briefly herein.

The system 100 includes a central processing unit (CPU 102) and a memory 104. The CPU 102 may be implemented as a microprocessor or part of a minicomputer or mainframe computer. The CPU 102 may be a conventional microprocessor chip, microcontroller, digital signal processor, or the like. Similarly, the memory 104 may be implemented by a variety of known technologies. The memory 104 may comprise random access memory (RAM), read-only memory, flash memory, or the like, or combinations thereof. The system 100 is not limited by the specific implementation of the CPU 102 and memory 104.

The system 100 also includes data storage 106, and conventional IO devices, such as a display 108, cursor control device 110, and keyboard 112. The data storage 106 may be implemented in a variety of forms, such as a hard disk drive, optical drive or the like. The display 108 is a conventional computer display having the necessary graphic resolution to allow satisfactory display of images, as will be described below. In a typical implementation, the display 108 is a color computer display. The cursor control 110 may be a joystick, mouse, trackball or the like. The keyboard 112 may be a conventional computer keyboard or may include custom keys to simplify the processes described herein.

Coupled to the system 100 is an imaging device 120. A number of different imaging devices are known in the art. Among them are conventional X-rays, computerized tomography (CT scanners), magnetic resonance imaging (MRI), positron emission tomography (PET), Single Photon-Emission Computed Tomography (SPECT), ultrasound imaging, or the like. One or more of these modalities may be used to provide imaging data to the system 100. The imaging data is processed and classified by Computer-Aided Detection (CAD) processor 122. The CAD processor 122 may detect and/or diagnose a VOI automatically or simply identify in segment certain regions in the image based on sets of rules established by the radiologist and/or surgeon. Examples of CAD processors are described, by way of example, in U.S. application Ser. No. 09/721,913, entitled CONVOLUTION FILTERING OF SIMILARITY DATA FOR VISUAL DISPLAY OF ENHANCED IMAGE, filed Nov. 24, 2000, now allowed, and U.S. application Ser. No. 09/722,063 entitled DYNAMIC THRESHHOLDING OF SEGMENTED DATA SETS AND DISPLAY OF SIMILARITY VALUES IN A SIMILARITY IMAGE, filed Nov. 24, 2000, now pending. These applications are assigned to the assignee of the present invention and are incorporated by reference in their entirety.

Particular imaging techniques, such as MRI, may scan a volume of tissue within a region of anatomical interest. Scan information or data corresponding to an anatomical volume under consideration may be transformed into or reconstructed as a series of planar images or image “slices.” For example, data generated during a breast MRI scan may be reconstructed as a set of 40 or more individual image slices. Any given image slice comprises an array of volume elements or voxels, where each voxel corresponds to an imaging signal intensity within an incremental volume that may be defined in accordance with x, y, and z axes. The z axis commonly corresponds to a distance increment between image slices, that is, image slice thickness.

Any given medical imaging technology may be particularly well suited for differentiating between specific types of tissues. A contrast agent administered to the patient may selectively enhance or affect the imaging properties of particular tissue types to facilitate improved tissue differentiation. For example, MRI may excel at distinguishing between various types of soft tissue, such as malignant and/or benign breast tumors or lesions that are contrast enhanced relative to healthy breast tissue in the presence of Gadolinium DPTA or another contrast agent.

Medical imaging techniques may generate or obtain imaging data corresponding to a given anatomical region at different times or sequentially through time to facilitate detection of changes within the anatomical region from one scan to another. Temporally varying or dynamic tissue dependent contrast agent uptake properties may facilitate accurate identification of particular tissue types. For example, in breast tissue, healthy or normal tissue generally exhibits different contrast agent uptake behavior over time than tumorous tissue. Moreover, malignant lesions generally exhibit different contrast agent uptake behavior than benign lesions (“Measurement and visualization of physiological parameters in contrast-enhanced breast magnetic resonance imaging,” Paul A. Armitage et al., Medical Imaging Understanding and Analysis, July 2001, University of Birmingham).

In general, at any particular time, the intensity of an imaging signal associated with any particular voxel depends upon the types of tissues within an anatomical region corresponding to the voxel; the presence or absence of a contrast agent in such tissues; and the temporal manners in which such tissues respond following contrast agent administration. In several types of breast MRI situations, normal or healthy tissue exhibits a background signal intensity in the absence of a contrast agent, while abnormal or tumorous tissue exhibits a low or reduced signal intensity relative to the background intensity. Thus, prior to contrast agent administration, abnormal tissue typically appears darker than normal tissue. In the presence of a contrast agent, lesions or certain types of abnormal tissue typically exhibit a time-dependent enhancement of imaging signal intensity relative to the background intensity.

In the above-referenced application entitled DYNAMIC THRESHHOLDING OF SEGMENTED DATA SETS, image slices are displayed in two dimensions as picture elements (i.e., pixels) that represent volume elements (i.e., voxels). In one exemplary embodiment described in that application, a caregiver, such as a radiologist, examines the imaged data and identifies one or more regions of interest, commonly referred to as a volume of interest (VOI). Based on the radiologist's analysis, certain voxels or discreet data elements may be identified as lesions. The CAD processor 122 utilizes a plurality of different measures of the physical characteristics of the selected discreet data elements and places them in a training set. Thereafter, other discreet data elements, representing additional voxels, are analyzed with respect to the training set to determine a similarity value. That is, the multiple physical characteristics of each discreet data element may be compared against the multiple physical characteristics of the training set and a similarity value determined based on this analysis. Those data elements having a sufficient similarity value may be displayed as a similarity image. In such an image, all discreet data elements or voxels meeting the requirement (i.e., having sufficient similarity to the training set) may be displayed. Use of this image classification allows the detection of areas that are similar to the training set, which has been identified by the radiologist as a lesion. This analysis may be extended to discreet data elements in regions other than the region surrounding the training set to identify metastasized cancer cells.

Returning again to FIG. 1, the CAD data, derived from the CAD processor 122, may be determined and image data provided to the data storage 106. A measurement module 130 is used to automatically or manually permit further characterizations of a VOI. That is, the measurement module 128 may be used to determine the location of a VOI (e.g., a lesion) with respect to anatomical or artificial landmarks. Further details of the measurement module 130 are provided below.

The system 100 also includes a volumetric modeling processor 130. As will be described in greater detail below, the volumetric modeling processor 130 is used in surgical planning to define a volume surrounding the lesion. This serves as a guide to surgeons that may be required to remove the lesion.

The system 100 also includes a network interface controller 132, which is coupled to a network 134. The network 134 may be any conventional form of network, such as a local area network (LAN), a wide area network (WAN), or the like. The network interface controller 132 may be selected based on the network type and the interface type. For example, in one embodiment, the network interface controller 132 may be an ether net controller. Alternatively, the network interface controller may be a USB interface, a dial-up modem or constructed in accordance with IEEE-1394 interface. The system 100 is not limited by the specific form of the network 134 nor the network interface controller 132.

The various components described above are coupled together by a bus system 136, which may include a data bus, address bus, control bus, power bus, and the like. For the sake of clarity, those various buses are illustrated in FIG. 1 as the bus system 136.

Those skilled in the art will recognize that many of the functional blocks illustrated in the functional block diagram of FIG. 1 may be implemented as standalone hardware or as a set of computer instructions stored in the memory 104 and executed by the CPU 102. For example, the measurement module 128 may be implemented as a set of software instructions executed by the CPU 102. Similarly, other elements, such as the CAD processor 122 and the volumetric modeling processor 130 may be implemented by hardware components, such as a digital signal processor, or maybe implemented as a set of software instructions stored in the memory 104 and executed by the CPU 102. However, each of these blocks performs a separate function and is thus illustrated in the functional block diagram of FIG. 1 as a separate element. However, the system 100 is not limited by the specific implementation of the various components.

The system 100 allows treatment of a patient and surgical planning to be carried out in an efficient and cost effective manner. The system 100 creates pre-treatment reports that identify the detected lesions, determine measurements of lesions in three dimensions, determine measurements of the location of lesions with respect to anatomical landmarks, and the calculation of a volume of tissue for each VOI that must be removed in a surgical procedure or treated in a breast-conserving non-surgical treatment. The pre-treatment report may be readily stored in the data storage 106, or stored in a location remote to the system 100, such as a central storage location. In this embodiment, the pre-treatment report and associated data may be transmitted to a central storage location via the network 134 (e.g., the LAN or (WAN), in a manner well understood by those skilled in the art.

The system 100 can be readily implemented in a variety of different computer architectures. In one embodiment, the data storage 106 is a mass storage unit associated with the system 100. However, those skilled in the art will appreciate that the data storage 106 is intended to encompass not only local storage, but mass storage that may be available on the network 130, such as the LAN, or delivered to the storage area 106 at a remote location via a virtual private network (VPN) or wide area network (WAN). The location and specific form of the data storage 106 may be selected based on the particular needs of the system 100. The system 100 is not limited by the specific form of the data storage 106 nor its location with respect to the other components of the system 100.

Indeed, in a distributed model, various components of the system 100 may be remotely located from each other. For example, the imaging device 120 may typically be located in a radiology department of a hospital while the components of the system 100 may be located within the radiology department of a hospital or in some other location within the hospital. In yet another exemplary embodiment, the system 100 need not be within the hospital at all. The imaging data may be provided to the system 100 as a data file stored on a data storage device, or as a data file stored on a CD-ROM or transmitted over, by way of example, the network 134.

Similarly, the CAD processor 122 may be located remotely from other components of the system 100. As described above, the CAD processor 122 detects and diagnoses lesions to thereby identify one or more VOIs.

In another exemplary embodiment, the surgeon and/or radiologist may be at a computer or terminal that may be remote from the system 100. For example, the patent application entitled SYSTEM AND METHOD FOR DISTRIBUTING CENTRALLY LOCATED PRE-PROCESSED MEDICAL IMAGE DATA TO REMOTE TERMINALS, describes a system in which the CAD portion (e.g., the CAD processor 122) is centrally located and the physician views pre-processed data from a remote terminal. A similar architecture could be applied to the system 100 to permit the physician to view the pre-treatment reports and/or post-treatment reports from a remote terminal. Distributed computing environments are well known in the art and can be readily applied to the system 100. Accordingly, the system 100 is not limited by any specific computer architecture or the requirement that the components listed in FIG. 1 be co-located.

Throughout this whole process, different physicians are interested in potentially different images and sets of data. MR studies often result in thousands of images. The radiologist then is responsible for analyzing the images and identifying tissues of interest, which may vary depending on the type of report. The report may also contain information to meet the recommendations in the American College of Radiology Breast Imaging and Reporting Data System (ACR BI-RADS®) Breast Imaging Atlas. This information may be chosen by the radiologist, or automatically computed for the identified tissues of interest. FIG. 1 illustrates a number of different reports that can be created and individually customized for report types, or for different physicians or both. This feature provides a mechanism to provide custom views of imaging results for the various physicians, while minimizing the effort of the radiologist to create these reports.

Although the techniques discussed herein use examples directed to evaluation of breast tumors, the techniques are more widely applicable to the evaluation of tissue for surgical planning purposes in general.

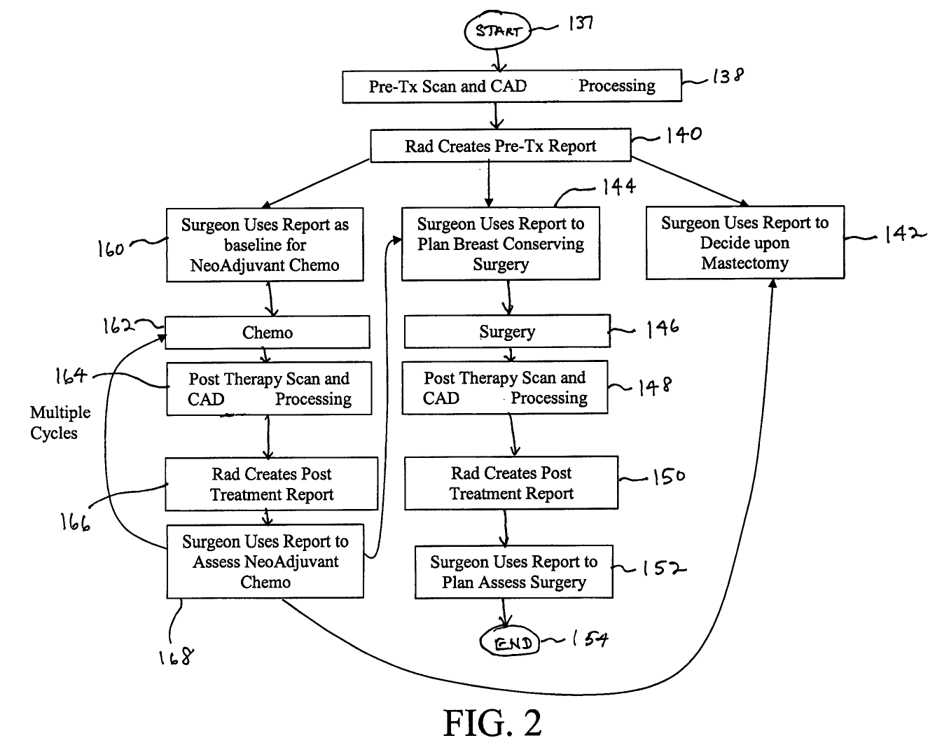

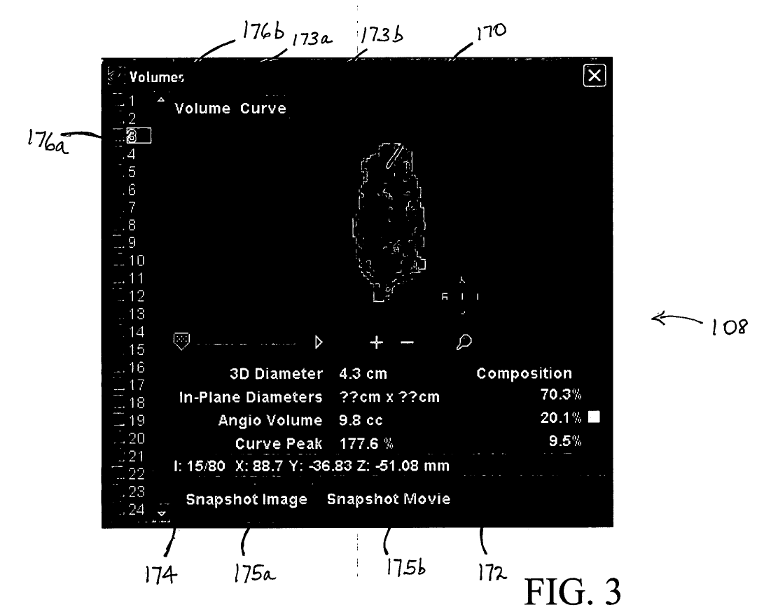

FIG. 2 illustrates a treatment planning and monitoring management workflow that may be readily implemented by the system 100. At a start 137, a patient is recommended for evaluation by the system and, in step 138, the imaging device 120 is used to generate the necessary images. In a breast evaluation, this may, by way of example, comprise forty or more image slices of each breast and may include pre-contrast image slices as well as post-contrast image slices after the introduction of a contrast agent, as-described above. These multiple images are used-by the CAD processor 122 to detect all VOIs. In step 140, the system 100 creates a pre-treatment report. As previously discussed, the various modes of imaging collected by the imaging device 120 are provided to the data storage 106. A caregiver, typically a radiologist, creates the pre-treatment report at step 140 by analyzing the imaged data and identifying all potentially malignant VOIs. This step may also include classifying the lesions according to some standard, such as the ACR BI-RADS. The classifications may be automatically computed, or manually specified by the radiologist. FIG. 3 illustrates an image of a VOI 170 shown on the display 108 (see FIG. 1) and the associated measurement data generated by the measurement module 128.

One skilled in the art will appreciate that medical image data, such as MRI data, typically includes a large number of images. For example, breast imaging often involves the administration of a contrast agent. In the moments following the administration of the contrast agent, a series of images, perhaps 100 or more, are obtained. In addition, images may be obtained from different orientations, such as a series of sagital images, a series of coronal images, and the like. Furthermore, those skilled in the art will appreciate that a typical MRI series contains a plurality of “slices” representing different image planes within the imaged portion of the patient anatomy. The system 100 automatically evaluates a large number of available images to select one or more images that best depict the VOI. Thus, the system advantageously analyzes a large number of images and selects the most appropriate images for inclusion in the report. This is a considerable savings in time from the conventional technique that requires the radiologist to manually evaluate all images to determine which few images to include in the report.

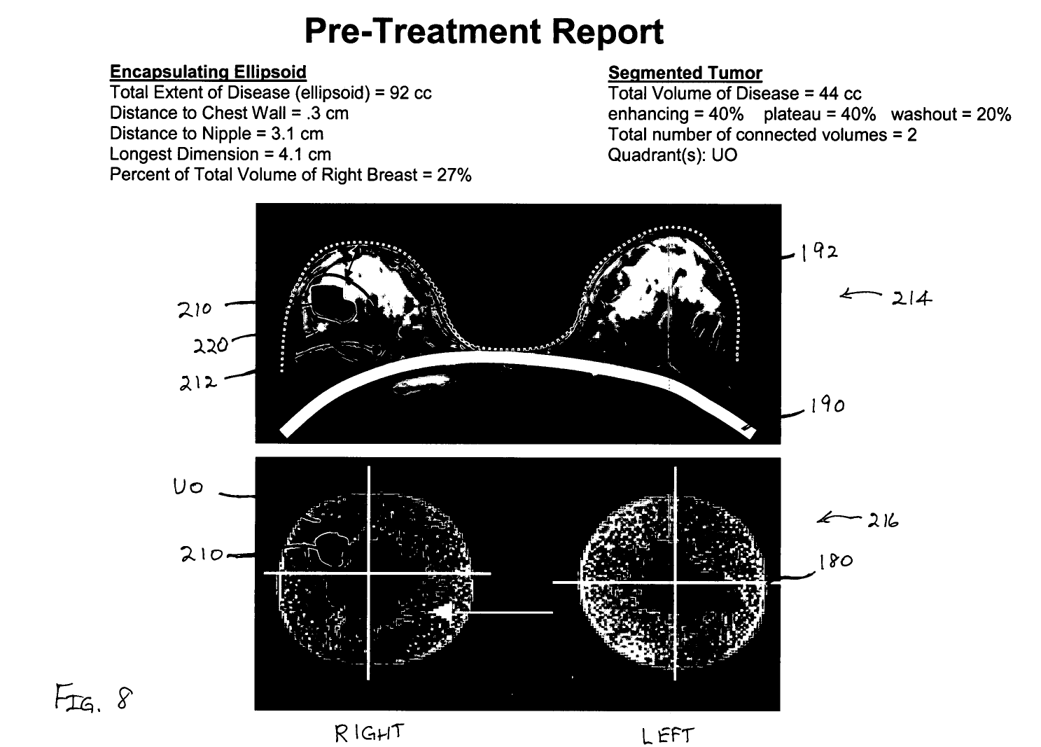

To illustrate the concept of automatic report generation, consider the image of FIG. 8, which is a one page pre-treatment report on a selected lesion. FIG. 8 includes 2 images selected from a superset of medical images for the particular patient. The report may include image identification information that permits the retrieval of original images or the evaluation of related images. For example, it may be desirable for a surgeon to evaluate multiple slices of a particular VOI to better understand the shape and position of a particular VOI.

The system 100 analyzes different slices to determine the slice with the largest cross-sectional area. The image having the largest cross-sectional area may be included as a selected image. In addition, the system 100 may evaluate a series of slices to determine a centroid for the selected VOI. In addition, the system 100 may evaluate multiple images to determine a volume surrounding the VOI. As previously noted, the surrounding volume may be characterized as an ellipsoid to assist the surgeon in surgical planning for possible removal of the VOI.

In one embodiment, the system 100 may select images based on the location of the VOI. This permits the selection of images that best illustrate the location of the VOI. As will be discussed in greater detail, the location may also be illustrated on a wire frame model.

In another embodiment, the images may be selected for inclusion in a report on the basis of size. That is, the system 100 may evaluate images to select one or more images that best illustrate the size of the VOI. The system 100 may also include one or more images based on both location and size.

As illustrated in FIG. 8, size and location information is calculated and displayed for the selected VOI. The system 100 automatically analyzes multiple images to determine data, such as the longest ellipsoid diameter or in-plane diameters. Thus, the system 100 automatically analyzes a large number of images and selects the best images to include in a report. The images selected may be determined on the basis of report type. For example, a surgeon may require selected images that best serve the purpose of surgical planning. The surgical planning report can include image views selected by the individual surgeon or specified in a predetermined report format. The report format and selected images may be determined by standards, such as the ACR BI-RADS. In another example, treatment planning may require different images and a different associated data than may be required for surgical planning. Accordingly, a treatment planning report type can include additional or different images and associated data that are most useful to the caregiver. A treatment planning report format can also be specified by the individual caregiver or specified in a predetermined report format. The report format and selected images may be determined by standards. All customized report formats, whether selected by individuals or using predetermined formats, can be stored in the data storage 106 (see FIG. 1) for future use in automatically generating subsequent reports using the stored formats.

In one aspect, the system 100 can be used as a surgical planning tool. Based on the pretreatment report generated at step 140, the surgeon may simply use the report to determine that a mastectomy is the most appropriate form of treatment, as shown in step 142.

However, in another aspect, the system 100 may be used not only for surgical planning, but for treatment in monitoring. For example, the surgeon may use he pre-treatment report generated at step 140 to plan breast conserving surgery at step 144. In step 146, the surgery is performed and, in step 148, post-therapy scanning and CAD processing occurs. That is, the system 100 may utilize the CAD processor 122 to monitor lesions or VOIs (e.g., the VOI 170 FIG. 3) following surgery.

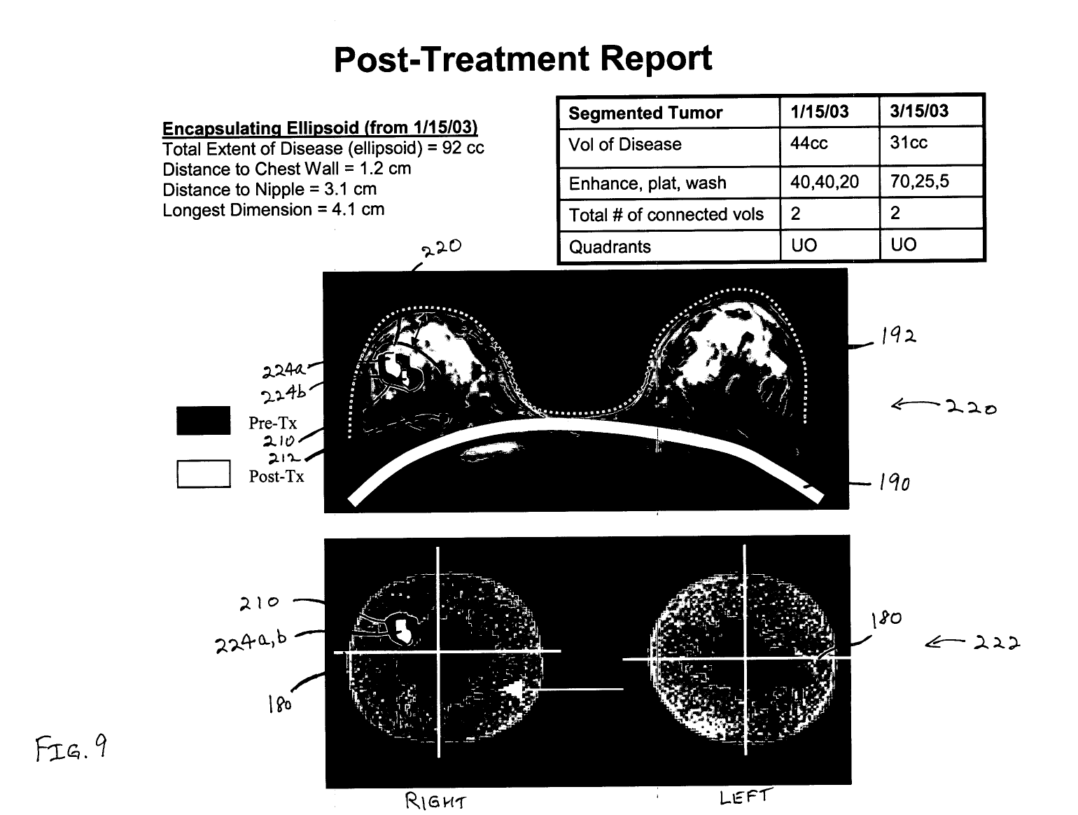

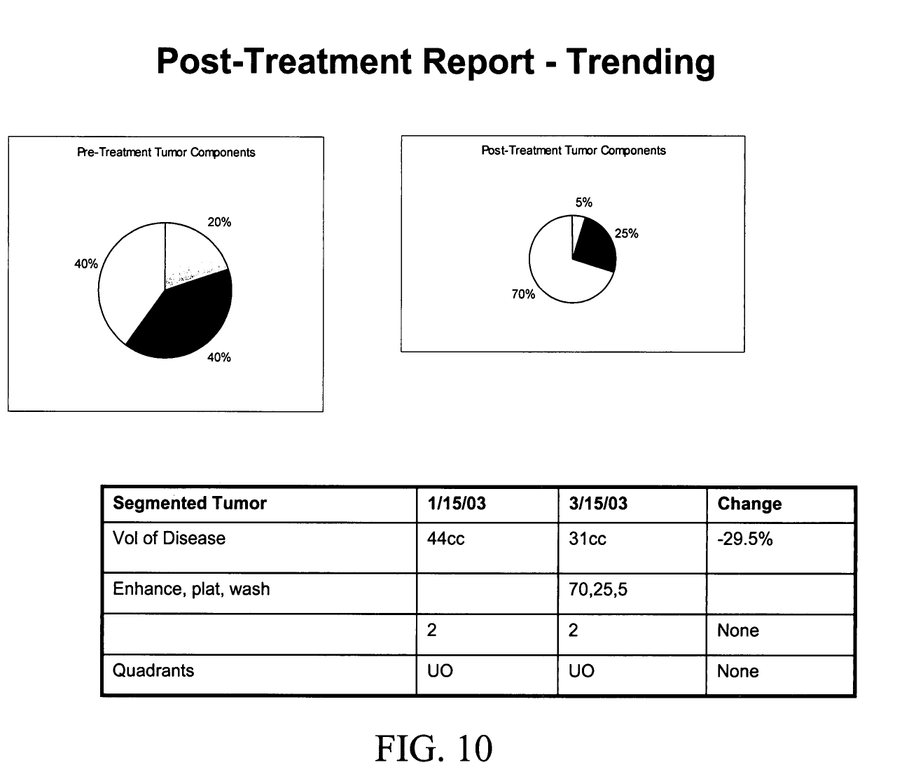

Following surgery, the system 100 creates a post-treatment report in step 150. An example of a post-treatment report is illustrated in FIGS. 9-10. Details of post-treatment reports are provided below. In step 152, the surgeon uses the report to plan or assess surgery and the process ends at 154. Those skilled in the art will appreciate that various stages of this process may be repeated as warranted.

It should be understood that the system 100 may used for surgical planning and treatment planning/monitoring using other treatment techniques. For example, new stages of treatment are constantly being developed by groups, such as the American Society of Breast Surgeons. For example, ablative and minimally invasive percutaneous excisional treatments for early stage of breast cancer are being investigative by various groups involved with breast cancer research. At this time, these techniques include ablation by laser, cryotherapy, microwave, and radio frequency. Percutaneous excision by rotational or vacuum-assisted devices is also being investigated. As can be appreciated by those skilled in the art, the system 100 may be used for pre-treatment and post-treatment reports for any type of surgical or treatment regimen. Thus, the system 100 is not limited by the specific surgical techniques described herein.

Returning again to step 140, in a third aspect of the system 100, the surgeon may use the pre-treatment report as a baseline for Neo-Adjuvant chemotherapy. It is well-known that chemotherapy and/or radiation therapy may be used to reduce the size of tumors prior to surgery. The advantage of the system 100 is that it can readily monitor progress of pre-operative treatment, such as a reduction in tumor size, and thereby give the surgeon the greatest amount of useful information regarding the size and location of tumors.

In step 160, the surgeon uses the report as the baseline for such treatment. In step 162, the chemotherapy is administered to the patient and, in step 164, post-therapy scan and CAD processing is performed. The CAD processor 122 is used in the manner described to monitor the detected tumors.

In step 166, the system 100 is used to create a post-treatment report. FIG. 9 illustrates an example of a post-treatment report. Additional data, such as post-treatment trending data, illustrated in FIG. 10, may also be generated for use by the surgeon. These reports and additional data are discussed in greater detail below. In step 168, the surgeon uses to post-treatment report to assess the Neo-Adjuvant chemotherapy treatment. The surgeon may elect to return to step 162 for additional chemotherapy treatment. Multiple cycles of chemotherapy and post-treatment scanning and reporting may be performed as deemed necessary by the surgeon.

Following one or more cycles of chemotherapy and post-therapy scanning and reporting, the surgeon may move to step 142 to perform a mastectomy, if warranted, or may move to step 144 to plan breast conserving surgery. In either event, the system 100 may be used following surgery to ensure that all suspect tissue has been removed. As previously discussed, positive margins are not uncommon. However, with the planning and monitoring processes provided by the system 100, the surgeon has an opportunity to plan the surgical procedure so as to minimize the chances of a positive margin. In addition, the CAD processor 122 can be used to readily identify positive margins if they should occur.

As previously indicated, FIG. 3 illustrates one example of an image created for the pre-treatment report. In addition to showing the VOI 170 on the display 108, the measurement module 128 (see FIG. 1) may be used to provide measurement data 172. The measurement module 128 may automatically perform measurements or may be used in conjunction with the cursor control 110 to permit manual measurements of the VOI 170. The measurement data includes the three-dimensional diameter of the VOI 170 as well as the length and width of the particular image slice being displayed on the display 108. The measurement module 128 also calculates the angio volume of the VOI 170. The angio volume indicates the portions of he tumor exhibiting angiogenesis.

In addition to measurement data, the display 108 provides data relating to curve peak, which is an indication of the percent enhancement with pre- and post-contrast data. As those skilled in the art will appreciate, tumor cells typically exhibit a rapid uptake of contrast agent and percent enhancement measurement is frequently used to indicate potentially cancerous lesions. In addition to rapid uptake of contrast agent, cancerous cells tend to demonstrate a sudden decrease or washout of the contrast agent. Thus, certain cells indicate a rapid uptake followed by a rapid washout of cells. Other cells indicate a rapid uptake but the percent enhancement tends to peak and form a plateau. Still other cells tend to have a rapid uptake of contrast agent within a short period of time and continue to show a persistent or continuous enhancement. The display 108 includes composition data that divides the cells within the VOI 170 into one of these subcategories. That is, in the example illustrated in FIG. 3, 70.3% of the data elements or voxels that make up the VOI 170 exhibit persistent enhancement behavior. The data in FIG. 3 also shows that 20.1% of the data elements in the VOI 170 exhibit plateau behavior; that is, there is a rapid uptake of the contrast agent causing an enhancement of the imaging followed by a plateau in which the percent enhancement remains substantially constant. Finally, the data displayed in FIG. 3 illustrates that 9.5% of the data elements in the VOI 170 exhibit washout characteristic behavior. Characterizing the initial rise and the delayed phase of the enhancement curve is also important in the BI-RADS classification. The physician can use this composition data to determine whether a VOI (e.g., the VOI 170 of FIG. 3) is a cancerous lesion or some noncancerous mass.

The data shown on the display 108 illustrates the volume of the VOI 170, which may be selected by selecting a volume selector tab 173a. The actual curves associated with the composition data, described above, may be shown on the display 108 by selecting the curve tab 173b. A data indicator 174 identifies the particular image slice in a collection of data. For example, as noted above, breast images for MRI may include 40 image slices for each breast, for a total of 80 images. In the example illustrated in FIG. 3, the image indicator identifies the particular image as the 15th slice out of 80. Those of ordinary skill the art will recognize that imaging techniques, such as MRI, result in a plurality of images. An MRI breast study may typically involve one pre-contrast series of images and 3-5 post-contrast series of images. Each series is composed of images representing slices of the breasts. The slices may be acquired as transverse, sagital, or coronal. Typically, the number of slices needed to image both breasts is between 60 and 150 images for transverse or coronal (since both breasts are shown in every image) and 150-250 slices for a sagital image. The data shown on the display 108 is one image slice in a series of 80 images.

A snapshot image control allows the physician to store the particular image and associated data within the data storage 106. Alternatively, the physician may select a snapshot movie control 175b to store data sequence in which the VOI 170 is rotated about an axis to allow a three-dimensional viewing of the VOI. The snapshot movie data may also be stored in the data storage 106.

A count indicator 176a and associated checkbox lists the number of VOIs that were detected by the CAD processor 122 (see FIG. 1). The first 24 VOIs may be selected using conventional curser control techniques. The remaining VOIs may be selected through the manipulation of a slide control 176b in a well known manner. In the example of FIG. 3, the VOI 170 is the third VOI detected by the CAD processor 122. The physician may check the checkbox accompanying the VOI to indicate that this is a likely tumor. Thus, the system provides a convenient technique for listing all VOIs that are suspicious or identified as tumors. All the data from the various VOIs in snapshot images and other data are stored in the data storage 106 to be used in a preparation of a pre-treatment report.

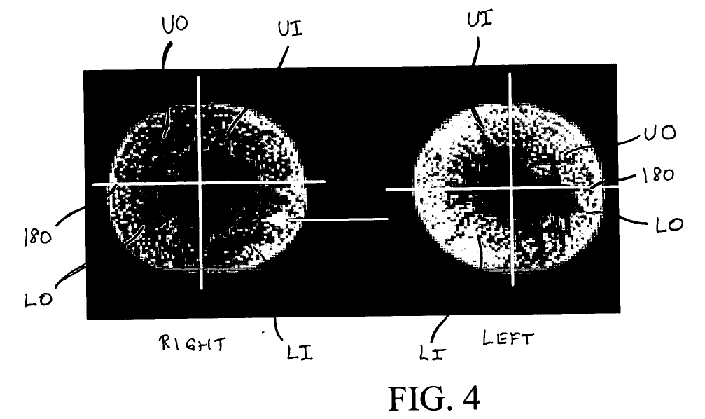

An example of the creation of a pre-treatment report is illustrated in FIGS. 4-8. The example treatment is directed to breast imaging and breast tumor detection, location, and monitoring. However, the principles of the system 100 can be readily extended to other tissue types and other anatomical locations. Thus, the system 100 is not limited to breast imaging.

The process of creating the pre-treatment report includes the identification of all VOIs and the likelihood of a particular VOI being a tumor. The identification and classification of a VOI is illustrated, by way of example, in FIG. 3 for a VOI of interest (i.e., a VOI that has been identified as a likely tumor).

Because a number of different images are created over a period of time, it is necessary to establish anatomical landmarks that may be used as registration references. Registration is the process of aligning two images for comparison. In the context of the present description pre-treatment and post-treatment images are registered so that the VOIs may be properly identified and located. Thus, the landmarks assist in registration to permit the identification and location of each VOI (e.g., the VOI 170 of FIG. 3). With breast imaging, the location of the nipple and location of the chest wall are commonly used anatomical landmarks. It should be recognized that nipple location and chest wall location are merely convenient recognizable landmarks. However, the use of any particular landmark is optional. For example, it may be useful to identify both nipple location and chest wall location, or only one. In addition, other landmarks, including artificial ones, may be used for rendering purposes or for calculating distances to precisely establish the location of any particular VOI. For example, surgically implanted clips may be used as landmarks. The advantage of the system 100 lies in its ability to accurately determine the location and size of VOIs and to provide the physician with techniques that allow treatment monitoring and/or surgical planning.

FIG. 4 illustrates-a coronal view of an MRI image in which a crosshairs 180 is positioned over the nipple to define four quadrants of each breast. The particular image illustrated in FIG. 4 is from an image slice in the mid-breast region. However, images closer to the surface of the breast readily identify the nipple and allow the system 100 to automatically position the crosshairs 180 on the nipple. Alternatively, the curser control 110 (see FIG. 1) may be used to manually place the crosshairs at the desired location. Once the position of the crosshairs has been fixed (either manually or automatically), that position is maintained throughout subsequent image slices to segment the breast into quadrants. The quadrants are typically identified as the upper inner and outer quadrants and lower inner and outer quadrants, for the left and right breasts. The separation between the left and right breasts are determined by the location of the quadrants defined by the crosshairs.

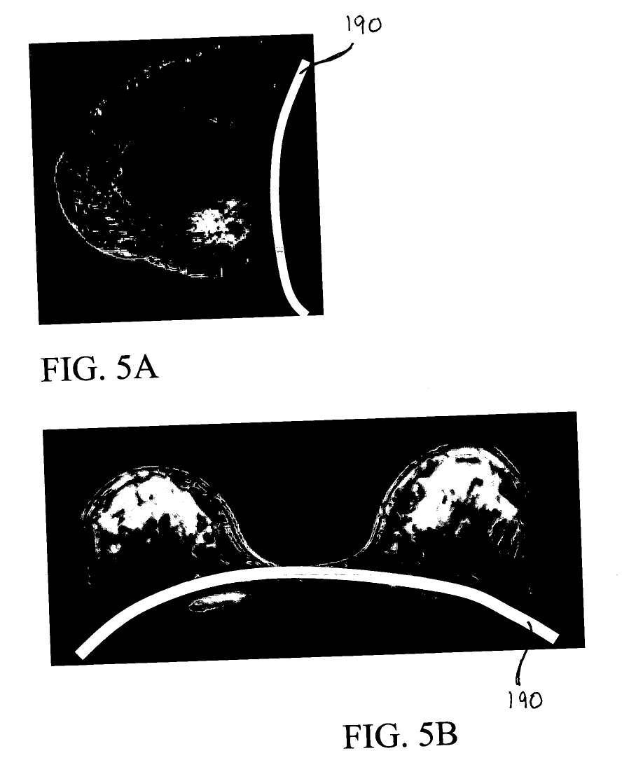

As a next step, the chest wall is identified in two separate views, illustrated in FIGS. 5A and 5B. FIG. 5A is a sagital view of one breast while FIG. 5B is a transverse axial view of both breasts. In FIGS. 5A and 5B, a chest wall 190 is identified and marked. In one embodiment, the chest wall 190 is automatically identified by the system 100 and marked as illustrated in FIGS. 5A-B. Alternatively, the chest wall 190 may be manually located and marked. In yet another alternative embodiment, the system 100 may automatically identify and mark the chest wall, but provide the option for overriding that determination if the physician desires. The chest wall 190 will serve as an anatomical landmark for future imaging.

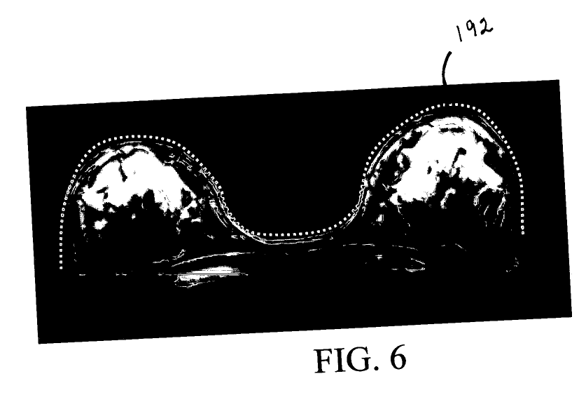

Finally, the system 100 identifies a skin surface 192 in three dimensions, as illustrated in FIG. 6. The system 100 can automatically detect the skin surface from the imaging data. The skin surface 192 is also used in the registration process.

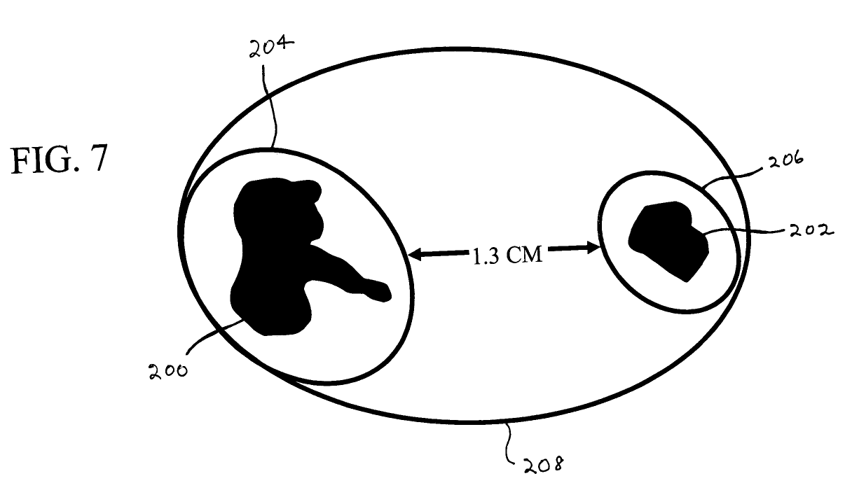

Part of the pre-treatment report is the generation of an area or volume indicator surrounding each VOI. FIG. 7 illustrates the generation of such indicators. The volumetric modeling module 130 functions to determine the volume of the breast, and the volume surrounding each VOI. The volume may be readily determined by analyzing the area of a lesion in each of multiple sequential image slices. Thus, the cross-sectional area in each image slice is determined and summed. In one embodiment, the actual volume of the lesion may be calculated. However, for surgical planning purposes, an area surrounding the volume of interest may be more useful.

In an exemplary embodiment, the volumetric modeling module 130 creates ellipsoid shapes surrounding each VOI (e.g., VOI 170 FIG. 3). The use of ellipsoid shape volumes is selected to correspond with the shape of tissue volume generally removed by surgeons when excising a lesion. However, those skilled in the art will appreciate that other shapes may be used by the volumetric modeling module 130 and that ellipsoid shapes are only one of many different modeling volumes. Accordingly, the present invention is not limited by the particular modeling volume selected for use by the volumetric modeling module 130.

The illustration of FIG. 7 shows two VOIs, which are identified as a VOI 200 and a VOI 202. The volumetric modeling module 130 (see FIG. 1) generates a ellipsoid 204 to surround the VOI 200 and an ellipsoid 206 to surround the VOI 202. In addition, the volumetric modeling module 130 generates a large ellipsoid 208, which encompasses both the VOI 200 and the VOI 202. For surgical planning purposes, the physician may consider removing each VOI (i.e., the VOI 200 and the VOI 202) separately. In this case, the ellipsoid 204 and the ellipsoid 206 are used to guide the surgeon in determining the volume of tissue to be removed to increase the likelihood that the entire lesion will be removed. If the spacing between the ellipsoids 204 and 206 is insufficient, the surgeon may choose to remove both VOIs (i.e., the VOI 200 and the VOI 202) together. In that event, the large ellipsoid 208 can be used to guide the surgeon in the removal of both VOIs. Thus, the system 100 provides data to the surgeon for planning purposes. In addition, the various ellipsoids may be used during an adjuvant chemotherapy regimen to monitor a reduction in the size of the lesions and later the surgical plan if appropriate. For example, the initial evaluation may have indicated the removal of breast tissue corresponding to the large ellipsoid 208. However, following adjuvant chemotherapy, the surgeon may alter the surgical plan to remove tissue corresponding to the smaller ellipsoids 204 and 206, respectively.

FIG. 8 illustrates an example of a pre-treatment report. The data used to generate the report is based on data from the imaging device 120, which has been processed by the CAD processor 122, the measurement module 128 and the volumetric modeling module 130. The resultant data and associated images may be convenient stored in the data storage 106.

In the pre-treatment report of FIG. 8, a large VOI 210 and a small VOI 212 are identified in first and second images 214 and 216. The image 214 is a transverse axial image in which the chest wall 190 and the skin surface 192 have been identified and marked. As noted above, the chest wall 190 and skin surface 192 are used for registration of subsequent images, such as those used to generate a post-treatment report. The image 216 is a coronal image illustrating the crosshairs 180. As can be readily seen from the image 216, the VOI 210 is in the upper outer (UO) quadrant of the right breast.

Those skilled in the art will recognize that the VOIs may not be visible in all images. For example, the transverse axial image 214 shows both the VOI 210 and the VOI 212 while the coronal image 216 shows only the VOI 210. The inability to view the VOI 212 in the image 216 may be due to the fact that the VOI is in a different image plane and thus not visible in the particular image plane selected as the image 216. The VOI 212 may also be hidden behind the VOI 210 and thus not visible in the coronal image 216. As can be readily seen in FIG. 8, the use of anatomical markers, such as the cross-hair 180 and the chest wall 190, aid the physician in locating the VOIs 210 and 212. FIG. 8 also illustrates an ellipsoid 220 generated by the volumemetric modeling module 130 (see FIG. 1).

The pre-treatment report also includes measurement data related to the VOIs 210 and 212 as well as measurement data related to the encapsulating ellipsoid 220. Data related to the VOIs 210 and 212 include, by way of example, the number of VOIs identified by the CAD processor 122 as well as the total volume of the VOIs. Location data within a particular quadrant is also indicated. The data related to the segmented tumor (i.e., the VOI 210 and the VOI 212) also includes the total volume of the VOIs. In the example illustrated in FIG. 8, the number of connected volumes (i.e., VOIs within the ellipsoid 220) use two and the total volume of the VOIs is 44 cubic centimeters (cc).

In addition, the pre-treatment report may include contrast imaging data. As previously discussed, contrast imaging may be used to differentiate between normal cells and cancer cells. The pre-treatment report illustrated in FIG. 8 includes data indicating the characteristic composition of the VOIs is also provided. In the example illustrated in FIG. 8, 40% of the data elements (i.e., voxels) associated with the VOI 210 and the VOI 212 exhibit persistent enhancement characteristics while 40% of the data elements exhibit plateau characteristics. Twenty percent of the elements associated with the VOI 210 and the VOI 212 exhibit washout characteristics.

For surgical planning purposes, the pre-treatment report also includes data relating to the ellipsoid 220 that surrounds the VOIs 210 and 212. In the example illustrated in FIG. 8, the ellipsoid 220 surrounds both the VOI 210 and the VOI 212. In another example, the system 100 may generate a separate ellipsoid around each VOI as illustrated in FIG. 7. Alternatively, the surgeon may determine that separate ellipsoids are warranted. Such decisions are generally based on the size and location of VOIs with respect to each other. The final decision as to the number of ellipsoids may be left to the discretion of the surgeon.

The data for the ellipsoid 220 may include the total volume of the ellipsoid as well as the percent of the ellipsoid volume compared to the total volume of the breast. The ellipsoid data also includes measurement data indicating, by way of example, the distance to the chest wall, the distance to the nipple, and the longest dimension of the ellipsoid 220. In an exemplary embodiment, the system 100 may provide a direction and distance from a landmark along the skin surface to the point at which the ellipsoid 220 (or the VOI: 210-212) are closest to the skin surface. For example, clock directions may be used to indicate a direction from the nipple (e.g., two o'clock) and a distance from the nipple (e.g., 5 centimeters) used to indicate to approximate position on the skin surface closest to the ellipsoid 220. This position may typically serve as the entry point for a surgical procedure to remove the tissue defined by the ellipsoid 220 (including the VOIs 210 and 212). In the example of FIG. 8, the ellipsoid 220 includes a volume of 95 ccs, which is 27% of the volume of the right breast. The total volume of the breast and the percent of that volume contained within-ellipsoids (e.g., the ellipsoid 220) are important factors for the surgeon to consider. The determination of which form of surgery to pursue may be made by the surgeon based on factors. For example, if the percent of total volume of the breast is relatively small, the surgeon may elect breast-conserving surgery. On the other hand, if the total volume contained within one or more ellipsoids (e.g., the ellipsoid 220), the surgeon may select a radical mastectomy. The ellipsoid data also indicates that the distance from the ellipsoid 220 to the chest wall is approximately 0.3 centimeters (cm) while the distance to the nipple is approximately 3.1 cm. The longest dimension of the ellipsoid 220 is 4.1 cm.

As previously discussed with respect to FIG. 2, the surgeon may elect to perform surgery based solely on the pre-treatment report. The surgery may be in the form of a mastectomy or breast conserving surgery, such as a lumpectomy. Alternatively, the surgeon may elect chemotherapy or other pre-surgical treatment in an effort to reduce the size of the tumor and, in turn, the volume that will be removed during the surgical procedure. Following one or more cycles of pre-surgical therapy (e.g., chemotherapy), the system 100 creates a post-treatment report. An example post-treatment report is illustrated in FIGS. 9 and 10. Because the size, shape and position of the breast may have changed from one imaging session to another, registration, or alignment, of the pre- and post-treatment volumes is required. For the sake of simplicity in the registration process, the breast may be modeled as a rigid body. In more complex analysis, the breast may be modeled as a non-rigid body, which requires additional registration steps. Morphing techniques, commonly used in computer graphics processing, may be used to alter the shape of the breast in a post-treatment report in an effort to more closely align the skin surface 192 in the pre-treatment and post-treatment images. However, such morphing techniques may inadvertently alter the measured volumes of VOIs by effectively compressing the image. It is not uncommon for the breast to be smaller in size following one or more rounds of chemotherapy. Morphing the post-treatment images may have the undesirable side effect of altering the volume measurements of VOIs.

The registration process also includes the registration of the cross-hair 180 as well as alignment of the chest wall 190 and the skin surface 192 in the various images. In one embodiment, the registration process may be automatically performed by the system 100. In an alternative embodiment, the coronal and transverse three dimensional views may be registered or aligned by the user using the cursor control 110 (see FIG. 1) to manipulate or align the images on the display 108.

Upon completion of the registration process, the original VOIs may be shown on the display from the pre-treatment report. In the example illustrated in FIG. 9, the VOI 210 and the VOI 212 are illustrated in images 220 and 222. In the example pre-treatment and post-treatment reports of FIGS. 8 and 10, respectively, it should be noted that the image 220 in the post-treatment report corresponds to the image 214 in the pre-treatment report (see FIG. 8) while the image 222 in the post-treatment report corresponds to the image 216 in the pre-treatment report.

In addition to showing the pre-treatment VOIs (i.e., the VOI 210 and the VOI 212), the post-treatment report illustrates VOIs following treatment (i.e., post-treatment VOIs). In the example of FIG. 10, the original VOI 210 has been reduced in size and fragmented into two separate VOIs, illustrated in the transverse image 220 in FIG. 10 as a VOI 224a and a VOI 224b. The image 220 also indicates that the adjuvant chemotherapy has eliminated the VOI 212. In the coronal image 222, the post-treatment VOIs overlap, resulting in an image that appears to show a single VOI 224a, b. Alternatively, the VOI 224b may be in a different image slice and thus not visible in the coronal image 222. The advantage of two views, such as the transverse image 220 and the coronal image 222 is that the surgeon may see multiple VOIs that overlap in one image or another.

The images illustrated in the present application are black and white or grayscale images. However, those skilled in the art will appreciate that the display 108 (see FIG. 1) is typically a color display. Accordingly, the system 100 takes advantage of color display capability by identifying different VOIs in different colors. For example, the pre-treatment VOIs 210 and 212 may be shown in one color in the pre-treatment report of FIG. 8-and the post-treatment-report of FIG. 9. The post-treatment VOIs 224a and 224b may be shown in the post-treatment report of FIG. 9 in a different color so as to indicate any change in the VOIs with greater clarity. The specific colors used for pre-treatment and post-treatment display of VOIs may be based on known factors, such as ease of visibility, good contrast between colors, and the like. The system 100 is not limited by any specific color selection. In an alternative embodiment, different graphic patterns may also be used to help differentiate between pre-treatment VOIs and post-treatment VOIs.

The post-treatment report illustrated in FIG. 9 also includes data regarding the segmented tumor and the encapsulating ellipsoid. In an exemplary embodiment, the post-treatment report includes tumor data from the pre-treatment report as well as post-treatment display of the same data. In the example of FIG. 9, the post-treatment report includes the number of identified VOIs, the location of the VOIs and the volume of the tumors based on the pre-treatment report and the post-treatment report. In addition, the percent of VOI tissue exhibiting persistent enhancement, plateau and washout characteristics, as described above, are shown on the report for both pre-treatment and post-treatment. Using the measured data provided in the post-treatment report combined with the images 220 and 22s in the post-treatment report, the surgeon can evaluate the success of the adjuvant chemotherapy.

The post-treatment report illustrated in FIG. 9 also provides the measurement data of the original ellipsoid 210.

The post-treatment report can also include trending data to provide the physician with further information regarding the progress of adjuvant chemotherapy. An example of trending data provided in the post-treatment report is illustrated in FIG. 10. The data in the example of FIG. 10 includes measurement data, such as that described above with respect to FIG. 9 as well as calculations regarding changes in data. For example, the volume of the disease (i.e., the tumor) in the pre-treatment report was 44 cc while the volume of the tumor in the post-treatment report is 31 cc. This indicates a 29.5% decrease in-volume. The-trending report in FIG. 10 can also show the change in the number of connected volumes (i.e., VOIs). An increase in the number of connected volumes may be the result of the cancer mass or volume breaking into multiple smaller pieces. The trending data can also be used to indicate, by way of example, lack of change due to the adjuvant chemotherapy treatment. In such case, the tumor size may be the same or larger.

The post-treatment report of FIG. 10 also includes graphical data to indicate the relative change of tumor components. As previously discussed, the tumor components may be classified by their ability to take up and washout image contrast agents. In the example illustrated in FIG. 10, the percentage of the tumor comprising cells exhibiting washout characteristics dropped from 20% to 5%. At the same time, the percentage of cells exhibiting plateau characteristics dropped from 40% to 25% while the percentage of cells exhibiting persistent enhancement characteristics rose from 40% to 70%. Changes in the composition of the tumor may serve as an indication of the effectiveness of the adjuvant chemotherapy. The characteristic data is also shown in the form of a pie chart in FIG. 10. In an alternative embodiment, the overall size of the pie chart may be altered to reflect the change in the overall tumor volume. Thus, the post-treatment pie chart is somewhat smaller to indicate the 29.5% reduction in the volume.

The physician advantageously use the system 100 to judge the efficacy of adjuvant chemotherapy treatment pre-operatively and may further use the information generated by the system for surgical planning purposes. The location, volume and shape of VOIs permit the surgeon to extract the tumor and a sufficient volume of surrounding tissue so as to minimize the occurrence of positive margins.

The system 100 may also be used post-operatively to monitor for positive margins. If additional surgery is required, the system 100 can generate the necessary reports for surgical planning and monitoring. Thus, the system provides great advantage to the physician pre- and post-operatively for monitoring purposes, for surgical planning purposes, and for analyzing the results of pre-operative therapy. Post-operatively, the system 100 can be used to detect positive margins or the reoccurrence of tumors in another region. The CAD system thereby increases the efficiency of the radiologist interpreting the scan, and the efficiency of the surgeon in managing cancer treatment whether through therapeutic treatment, surgery, or both.

The flexible system architecture allows efficient integration into hospital computer systems and hospital workflow. Improvements in efficiency and ease in integration into existing medical systems provides operational and economic advantages as well as increased technological capabilities.

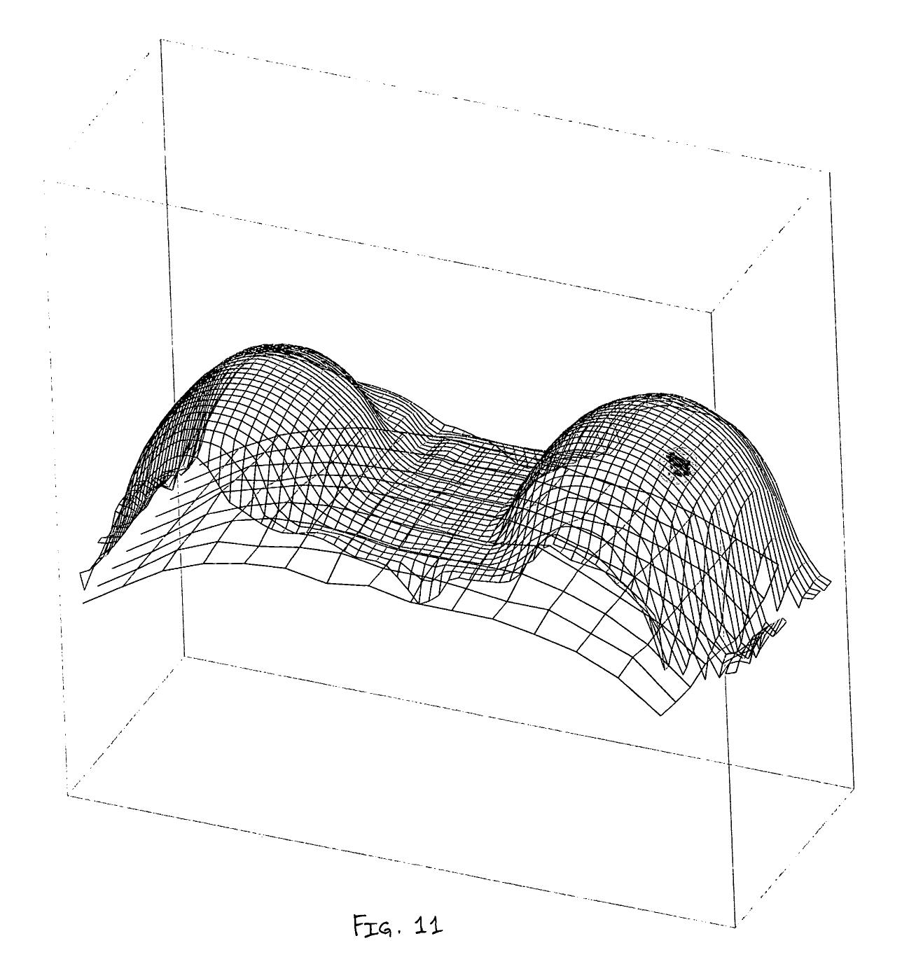

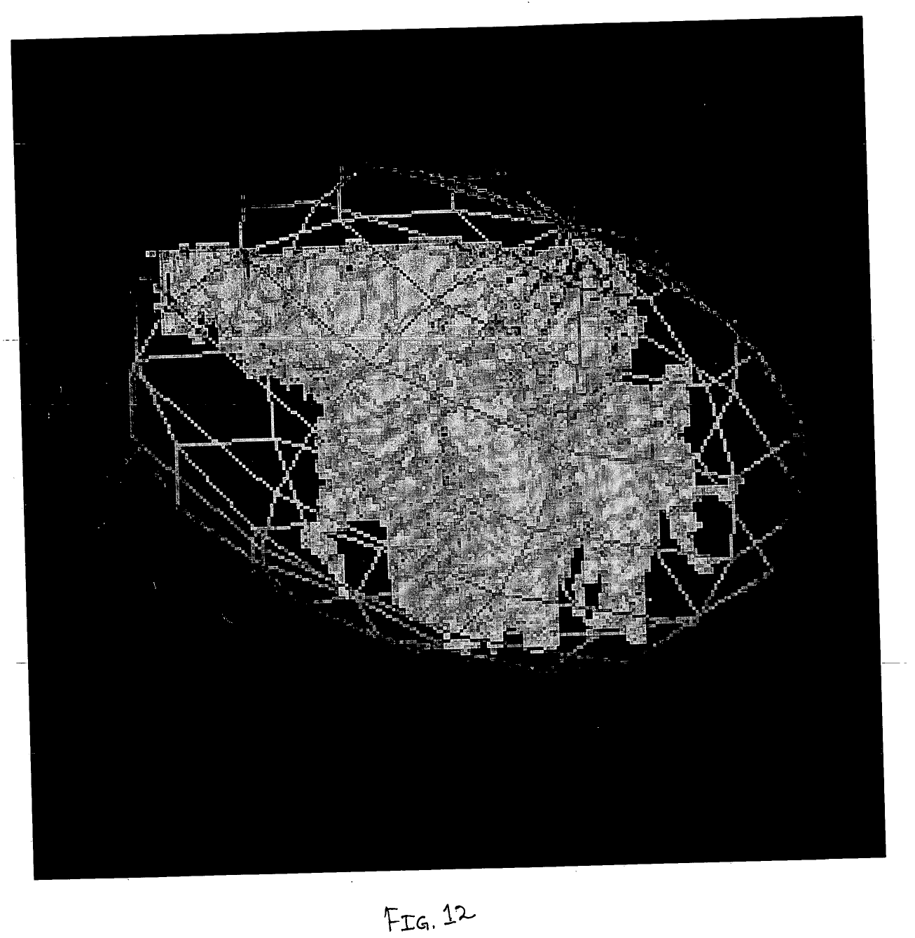

The images shown herein are actual MRI images of breast tissue with volumetric modeling to illustrate the location and size of tumors. In an alternative embodiment, the volumetric modeling module 130 (see FIG. 1) may use wire-frame modeling techniques, well known in the art of three-dimensional graphics processing, to illustrate the outline of the breast and landmarks, such as the nipple, chest wall, and skin surface. FIGS. 11-12 illustrate the use of wire frame models. As best illustrated in FIG. 11, the skin surface of the breast is illustrated as a wire frame model. Landmarks, such as the nipples, may be shown in model form as well. A VOI and surrounding ellipsoids can also be illustrated within the figure. The advantage of a wire frame model, such FIG. 11, is that it eliminates many of the artifacts present in a typical MR image. Thus, the use of wire frame modeling eliminates the visual artifact that may be associated with the MRI image data and allows a clear view of the VOI with respect to the wire-frame model.

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled” , to each other to achieve the desired functionality.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

1. A method for generating a medical planning report for a patient, comprising:
performing medical imaging test on a patient to thereby generate medical image data;
identifying landmarks in the medical image data;
identifying a lesion in the medical image data; and
generating data related to the identified lesion wherein the data is used to evaluate a medical plan for the patient.

2. The method of claim 1 wherein identifying a lesion comprises identifying a plurality of lesions.

3. The method of claim 1 wherein the medical plan is a surgical treatment planning report generated at a first time prior to treatment, and the generated data related to the identified lesion is used as a pre-treatment report.

4. The method of claim 1 wherein the medical plan is a medical treatment planning report generated at a first time prior to treatment, and the generated data related to the identified lesion is used as a pre-treatment report.

5. The method of claim 1, further comprising generating a report related to the identified lesion wherein the report includes at least one additional data element selected from a group of data elements comprising location data, distance from a landmark data, size data, volume data, enhancement composition data, and morphological indicators data.

6. The method of claim 1, further comprising generating a report related to the identified lesion wherein the report includes data conforming to report standards established by ACR BI-RADS.

7. The method of claim 1 wherein the medical image data comprises a plurality of individual images of the identified lesion, the method further comprising selecting ones of the plurality of images to include in a report related to the identified lesion.

8. The method of claim 7 wherein the selected ones of the plurality of images to include in the report are selected on the basis of a report type.

9. The method of claim 8 wherein the report type is selected from a group of report types comprising selected one of a surgical planning report type and a medical treatment planning report type.

10. The method of claim 7 wherein the selected ones of the plurality of images to include in the report are selected on the basis of lesion location within the patient.

11. The method of claim 7 wherein the selected ones of the plurality of images to include in the report are selected on the basis of lesion size.

12. The method of claim 11 wherein the lesion size is determined by calculating a volume of interest (VOI) surrounding the identified lesion.

13. The method of claim 1 wherein the identified landmarks are anatomical landmarks.

14. The method of claim 1 wherein the identified landmarks are artificial landmarks.

15. The method of claim 1 wherein the generated data comprises position data indicating a position of the identified lesion with respect to an identified landmark.

16. The method of claim 1 wherein the generated data comprises volume data indicating a volume size encapsulating the identified lesion.

17. The method of claim 16 wherein the volume data indicates a volume of an ellipsoid encapsulating the identified lesion.

18. The method of claim 16 wherein the generated data comprises volume data indicating a volume size encapsulating the identified lesion and a volume calculation for the anatomical structure in which the lesion is found.

19. The method of claim 1 for use in treatment of a breast lesion wherein the generated data comprises volume data indicating a volume size encapsulating the identified lesion and a volume calculation for the breast in which the lesion is found, the method further comprising calculating a proportion of the breast volume incorporated in the volume size encapsulating the identified lesion.

20. The method of claim 1 wherein the patient receives treatment of the identified lesion, the method further comprising:
at a time following the treatment, performing medical imaging test on the patient to thereby generate additional medical image data;
determining a location of the identified lesion in the additional medical image data; and
generating data related to differences in the identified lesion between the medical image data and the additional medical image data.

21. The method of claim 20 wherein the medical plan is a treatment planning report generated at a first time prior to treatment, and the generated data related to differences in the identified lesion between the medical image data and the additional medical image data comprises a post-treatment report used to evaluate the effectiveness of the treatment.

22. The method of claim 20 wherein generating data related to differences comprises performing a registration operation on the medical image data and the additional medical image data.

23. The method of claim 22 wherein registering comprises using identified anatomical landmarks, artificial landmarks, or a combination of anatomical landmarks and artificial landmarks in the medical image data and the additional medical image data.

24. The method of claim 22, further comprising:
determining volume data of a volume encapsulating the identified lesion in the medical image data;
determining volume data of a-volume encapsulating-the identified lesion in the additional medical image data; and
performing the registration operation comprises using the determined volume data in the medical image data and the determined volume data in the additional medical image data.

25. The method of claim 22 wherein performing a registration operation comprises accepting user input to manually register the medical image data and the additional medical image data.

26. The method of claim 22 wherein performing a registration operation is automatically performed between the medical image data and the additional medical image data.

27. The method of claim 20 wherein the patient receives additional treatment of the identified lesion, the method further comprising:
at a time following the additional treatment, performing medical imaging test on the patient to thereby generate subsequent medical image data;
determining a location of the identified lesion in the subsequent medical image data; and
generating data related to differences in the identified lesion between the additional medical image data and the subsequent medical image.

28. A method for generating a medical report for a patient, comprising:
performing medical imaging test on a patient to thereby generate medical image data;
identifying a lesion in the medical image data;
generating data related to the identified lesion to thereby generate a medical plan for the patient;
at a time subsequent to the execution of the medical plan for the patient, performing additional medical imaging test on the patient to-thereby generate additional medical image data;
registering the medical image data and the additional medical image data;
determining a location of the identified lesion in the additional medical image data; and
generating data related to differences in the identified lesion between the medical image data and the additional medical image data.

29. The method of claim 28 wherein registering comprises using identified anatomical landmarks, artificial landmarks, or a combination of anatomical landmarks and artificial landmarks in the medical image data and the additional medical image data.

30. The method of claim 28, further comprising:
determining volume data of a volume encapsulating the identified lesion in the medical image data; and
determining volume data of a volume encapsulating the identified lesion in the additional medical image data, wherein registering comprises using the determined volume data in the medical image data and the determined volume data in the additional medical image data.

31. The method of claim 28 wherein registering comprises accepting user input to manually register the medical image data and the additional medical image data.

32. The method of claim 28 wherein registering comprises automatically registering the medical image data and the additional medical image data.

33. A computer-readable medium for generating a medical report for a patient, comprising computer instructions that cause a processor to perform the steps of:
receiving medical image data for the patient;
identifying landmarks in the medical image data;
identifying a lesion in the medical image data; and
generating data related to the identified lesion wherein the data is used to evaluate a medical plan for the patient.

34. The computer-readable medium of claim 33 wherein identifying a lesion comprises identifying a plurality of lesions.

35. The computer-readable medium of claim 33 wherein the medical plan is a surgical treatment planning report generated at a first time prior to treatment, and the generated data related to the identified lesion is used as a pre-treatment report.

36. The computer-readable medium of claim 33 wherein the medical plan is a medical treatment planning report generated at a first time prior to treatment, and the generated data related to the identified lesion is used as a pre-treatment report.

37. The computer-readable medium of claim 33, further comprising generating a report related to the identified lesion wherein the report includes data conforming to report standards established by ACR BI-RADS.

38. The computer-readable medium of claim 33 wherein the medical image data comprises a plurality of individual images of the identified lesion, the computer-readable medium further comprising computer instructions that cause the processor to perform the step of selecting ones of the plurality of images to include in a report related to the identified lesion.

39. The computer-readable medium of claim 38 wherein the selected ones of the plurality of images to include in the report are selected on the basis of a report type.

40. The computer-readable medium of claim 38 wherein the selected ones of the plurality of images to include in the report are selected on the basis of lesion location within the patient.

41. The computer-readable medium of claim.38 wherein the selected ones of the plurality of images to include in the report are selected on the basis of lesion size.

42. The computer-readable medium of claim 41 wherein the lesion size is determined by calculating a volume of interest (VOI) surrounding the identified lesion.

43. The computer-readable medium of claim 33 wherein the identified landmarks comprise anatomical landmarks, artificial landmarks, or a combination of anatomical landmarks and artificial landmarks in the medical image data.

44. The computer-readable medium of claim 33 wherein the generated data comprises position data indicating a position of the identified lesion with respect to an identified landmark.

45. The computer-readable medium of claim 33 wherein the generated data comprises volume data indicating a volume size encapsulating the identified lesion.

46. The computer-readable medium of claim 45 wherein the volume data indicates a volume of an ellipsoid encapsulating the identified lesion.

47. The computer-readable medium of claim 45 wherein the generated data comprises volume data indicating a volume size encapsulating the identified lesion and a volume calculation for the anatomical structure in which the lesion is found.

48. The computer-readable medium of claim 33 for use in medical treatment of a breast lesion wherein the generated data comprises volume data indicating a volume size encapsulating the identified lesion and a volume calculation for the breast in which the lesion is found, the computer-readable medium further comprising computer instructions that cause the processor to perform the step of calculating a proportion of the breast volume incorporated in the volume size encapsulating the identified lesion.

49. The computer-readable medium of claim 33 wherein the patient receives treatment of the identified lesion, the computer-readable medium further comprising computer instructions that cause a processor to perform the steps of:
at a time following the treatment, receiving additional medical image data related to the treatment for the patient;
determining a location of the identified lesion in the additional medical image data; and
generating data related to differences in the identified lesion between the medical image data and the additional medical image data.

50. The computer-readable medium of claim 49 wherein the medical plan is a treatment planning report generated at a first time prior to treatment, and the generated data related to differences in the identified lesion between the medical image data and the additional medical image data is a post-treatment report used to evaluate the effectiveness of the treatment.

51. The computer-readable medium of claim 49 wherein generating data related to differences comprises performing a registration operation on the medical image data and the additional medical image data.

52. The computer-readable medium of claim 51, wherein registering comprises using identified anatomical landmarks, artificial landmarks, or a combination of anatomical landmarks and artificial landmarks in the medical image data and the additional medical image data.

53. The computer-readable medium of claim 49, further comprising computer instructions that cause a processor to perform the steps of:
determining volume data of a volume encapsulating the identified lesion in the medical image data;
determining volume data of a volume encapsulating the identified lesion in the additional medical image data; and
performing a registration operation using the determined volume data in the medical image data and the determined volume data in the additional medical image data.

54. The computer-readable medium of claim 51 wherein performing a registration operation comprises accepting user input to manually register the medical image data and the additional medical image data.

55. The computer-readable medium of claim 51 wherein performing a registration operation is automatically performed between the medical image data and the additional medical image data.

56. The computer-readable medium of claim 49 wherein the patient receives additional treatment of the identified lesion, the computer-readable medium further comprising computer instructions that cause the processor to perform the steps of:
at a time following the additional treatment, receiving subsequent medical image data related to the additional treatment for the patient;
determining a location of the identified lesion in the subsequent medical image data; and
generating data related to differences in the identified lesion between the additional medical image data and the subsequent medical image.

57. A computer-readable medium for generating a medical report for a patient, comprising computer instructions that cause a processor to perform the steps of:
receiving medical image data for the patient;
identifying a lesion in the medical image data;
generating data related to the identified lesion to thereby generate a medical plan for the patient;
at a time subsequent to the execution of the medical plan for the patient, receiving additional medical image data related to the executed medical plan for the patient;
registering the medical image data and the additional medical image data;
determining a location of the identified lesion in the additional medical image data; and
generating data related to differences in the identified lesion between the medical image data and the additional medical image data.

58. The computer-readable medium of claim 57 wherein registering comprises using identified anatomical landmarks, artificial landmarks, or a combination of anatomical landmarks and artificial landmarks in the medical image data and the additional medical image data.

59. The computer-readable medium of claim 57, further comprising:
determining volume data of a volume encapsulating the identified lesion in the medical image data; and
determining volume data of a volume encapsulating the identified lesion in the additional medical image data, wherein registering comprises using the determined volume data in the medical image data and the determined volume data in the additional medical image data.

60. The computer-readable medium of claim 57 wherein registering comprises accepting user input to manually register the medical image data and the additional medical image data.

61. The computer-readable medium of claim 57 wherein registering comprises automatically registering the medical image data and the additional medical image data.

62. An apparatus for generating a medical report for a patient comprising:
an input interface configured to receive medical image data for the patient;
a data structure configured to store the medical image data; and
a processor configured to:
identify landmarks in the medical image data;
identify a lesion in the medical image data; and
generate data related to the identified lesion wherein the data is used to evaluate a medical plan for the patient.

63. The apparatus of claim 62 wherein the processor is configured to identify a plurality of lesions.

64. The apparatus of claim 62 wherein the processor is further configured to generate a report related to the identified lesion wherein the report includes data conforming to report standards established by ACR BI-RADS.

65. The apparatus of claim 62 wherein the stored medical image data comprises a plurality of individual images of the identified lesion, the processor being further configured to select ones of the plurality of images to include in a report related to the identified lesion.

66. The apparatus of claim 65 wherein the processor selects ones of the plurality of images to include in the report on the basis of lesion location within the patient.

67. The apparatus of claim 65 wherein the processor selects ones of the plurality of images to include in the report on the basis of lesion size.

68. The apparatus of claim 67 wherein the processor is configured to determine lesion size by calculating a volume of interest (VOI) surrounding the identified lesion.

69. The apparatus of claim 62 wherein the landmarks identified by the processor comprise anatomical landmarks, artificial landmarks, or a combination of anatomical landmarks and artificial landmarks in the medical image data.

70. The apparatus of claim 62 wherein the processor is further configured to generate position data indicating a position of the identified lesion with respect to an identified landmark.

71. The apparatus of claim 62 wherein the processor is further configured to generate volume data indicating a volume size encapsulating the identified lesion.

72. The apparatus of claim 71 wherein the processor is configured to generate wherein the volume data indicating a volume of an ellipsoid encapsulating the identified lesion.

73. The apparatus of claim 62 wherein the processor is further configured to generate volume data indicating a volume size encapsulating the identified lesion and a volume calculation for the anatomical structure in which the lesion is found.

74. The apparatus of claim 62 for use in treatment of a breast lesion wherein the processor generates volume data indicating a volume size encapsulating the identified lesion and a volume calculation for the breast in which the lesion is found, the processor being further configured to calculate a proportion of the breast volume incorporated in the volume size encapsulating the identified lesion.

75. The apparatus of claim 62 wherein the patient receives treatment of the identified lesion, the processor being further configured to:
at a time following the treatment, receiving additional medical image data for the patient;
determine a location of the identified lesion in the additional medical image data; and
generate data related to differences in the identified lesion between the medical image data and the additional medical image data.

76. The apparatus of claim 75 wherein the medical plan is a treatment planning report generated at a first time prior to treatment, and the processor generates data related to differences in the identified lesion between the medical image data and the additional medical image data as a post-treatment report used to evaluate the effectiveness of the treatment.

77. The apparatus of claim 75 wherein the processor is further configured to generate data related to differences by performing a registration operation on the medical image data and the additional medical image data.

78. The apparatus of claim 77 wherein the processor performs registration using identified anatomical landmarks, artificial landmarks, or a combination of anatomical landmarks and artificial landmarks in the medical image data and the additional medical image data.

79. The apparatus of claim 75 wherein the processor is further configured to:
determine volume data of a volume encapsulating the identified lesion in the medical image data;
determine volume data of a volume encapsulating the identified lesion in the additional medical image data; and
perform a registration operation using the determined volume data in the medical image data and the determined volume data in the additional medical image data.

80. The apparatus of claim 75, further comprising a user input device wherein the processor is further configured to perform a registration operation comprises accepting data from the user input device to manually register the medical image data and the additional medical image data.

81. The apparatus of claim 75 wherein the processor is further configured to automatically perform a registration operation between the medical image data and the additional medical image data.

82. The apparatus of claim 75 wherein the patient receives additional treatment of the identified lesion, processor being further configured to:
at a time following the additional treatment, receiving subsequent medical image data for the patient related to the additional treatment;
determine a location of the identified lesion in the subsequent medical image data; and
generate data related to differences in the identified lesion between the additional medical image data and the subsequent medical image.

83. An apparatus for generating a medical plan for a patient, comprising:
an input interface to receive medical image data for the patient prior to the execution of the medical plan and to receive additional medical image data for the patient at a time subsequent to the execution of the medical plan;
a data structure to store the medical image data and the additional medical image data; and
a processor configured to:
identify a lesion in the medical image data; and
generate data related to the identified lesion to thereby permit the development of the medical plan for the patient;
register the medical image data and the additional medical image data;
determine a location of the identified lesion in the additional medical image data; and
generate data related to differences in the identified lesion between the medical image data and the additional medical image data.

84. The apparatus of claim 83 wherein the processor performs the registration using identified anatomical landmarks, artificial landmarks, or a combination of anatomical landmarks and artificial landmarks in the medical image data and the additional medical image data.

85. The apparatus of claim 83 wherein the processor is further configured to:
determine volume data of a volume encapsulating the identified lesion in the medical image data; and
determine volume data of a volume encapsulating the identified lesion in the additional medical image data, wherein the processor performs the registration using the determined volume data in the medical image data and the determined volume data in the additional medical image data.

86. The apparatus of claim 83, further comprising a user input device wherein the processor is further configured to perform a registration operation comprises accepting data from the user input device to manually register the medical image data and the additional medical image data.

87. The apparatus of claim 83 wherein the processor is further configured to automatically perform a registration operation between the medical image data and the additional medical image data.
